(19) United States
(12) Patent Application Publication
(10) Pub. No.: US 2006/0067880 A1
(43) Pub. Date: Mar. 30, 2006

(54) Labeled creatine ethyl ester hydrochloride

(76) Inventor: Lowell J. Lawrence, Lexington, KY (US)

Correspondence Address: KING & SCHICKLI, PLLC, 247 NORTH BROADWAY, LEXINGTON, KY, 40507, US

(21) Appl. No.: 11/075,177

(22) Filed: Mar. 8, 2005

Related U.S. Application Data
(60) Provisional application No. 60/613,858, filed on Sep. 28, 2004.

(51) Int. Cl. A61K 51/00; C07C 279/10

(52) U.S. Cl. 424/1.11; 560/168

(57) ABSTRACT

The present invention relates to various radiolabeled creatine ethyl ester hydrochloride compounds. Any carbon, hydrogen or nitrogen atom of the compounds may be radiolabeled using ¹⁴C, ¹³C, ²H, ³H or ¹⁵N atoms.

This application claims the benefit of U.S. Provisional Patent Application Ser. No. 60/613,858 filed 28 Sep. 2004.

### TECHNICAL FIELD

The present invention relates to radiolabeled creatine ethyl ester hydrochloride.

### BACKGROUND OF THE INVENTION

Creatine ethyl ester hydrochloride is a dietary supplement known to be useful to increase muscle mass and to enhance muscle performance. It is also used in emergency applications in the treatment of neuromuscular disorders. Published U.S. Patent Application U.S. 2003/0212136 A1 of Vennerstrom and Miller describes the composition of matter creatine ethyl ester hydrochloride and its method of production.

Creatine ethyl ester hydrochloride has been known since at least 1922. Creatine ethyl ester hydrochloride is made by reacting creatine with ethanol in the presence of hydrochloric acid.

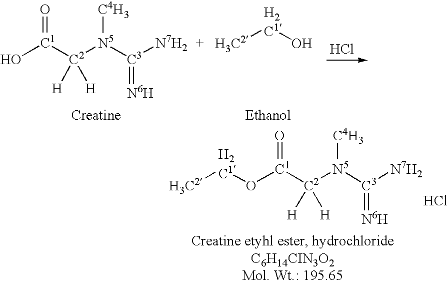

Creatine ethyl ester hydrochloride rapidly dissociates at physiological pH into ethanol and creatine. Creatine is present in the muscular tissue of many vertebrates. In humans creatine is produced in the liver and the kidneys. Ingestion of creatine has been known to increase muscle mass since at least 1992.

For purposes of study and testing it is desirable to know exactly how the ethanol and creatine moieties of creatine ethyl ester hydrochloride are being absorbed, distributed, metabolized and eliminated in the body. In order to achieve this end and allow researchers to independently follow both moieties through the metabolic process, individual carbon, hydrogen and/or nitrogen atoms at particular positions in the chemical structure may be labeled with ¹³C, ¹⁴C, ³H, ²H and/or ¹⁵N radioactive or stable isotopic atoms. The present invention relates to various creatine ethyl ester hydrochloride compounds labeled with these radioactive or stable isotopes.

### SUMMARY OF THE INVENTION

The present invention relates to compounds having the chemical formula

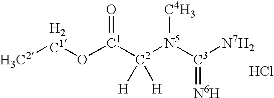

wherein the carbon at any one of the C-1′, C-2′, C-1, C-2, C-3 or C-4 positions is a ¹⁴C-labeled carbon;

or wherein the carbon at any one of the C-1′, C-2′, C-1, C-2, C-3 or C-4 positions is a ¹³C-labeled carbon;

or wherein a hydrogen atom connected to any one of the C-1′, C-2′, C-2 or C-4 carbons is an ²H-labeled hydrogen atom;

or wherein a hydrogen atom connected to any one of the C-1′, C-2′, C-2 or C-4 carbons is an ³H-labeled hydrogen atom;

or wherein a nitrogen atom at any one of the N-5, N-6 or N-7 positions is a ¹⁵N-labeled nitrogen atom.

### DETAILED DESCRIPTION OF THE INVENTION

A general procedure for the preparation of creatine ethyl ester hydrochloride will now be described.

Two ml of anhydrous ethanol is diluted with 67 μl of a 9:1 mixture of ethanol and water and stirred under an Argon atmosphere. To the mixture is added acetyl chloride (164 μl, 2.30 mmol) and the solution stirred for 5 minutes. The solution is heated to 40 degrees C. and creatine (200 mg, 1.53 mmol) is added to the stirring mixture. The mixture is stirred overnight at 40 degrees C. The creatine ethyl ester hydrochloride is recovered by cooling the mixture to 0 degrees C., filtering the white precipitate and washing it with a minimum quantity of cold ethanol. The material can be stored under anhydrous conditions at −15 degrees C.

Creatine ethyl ester hydrochloride, 3, can be prepared from appropriately labeled creatine and ethanol in various stable and radioactive forms following the general procedure described above.

In accordance with the present invention, creatine ethyl ester hydrochloride may be prepared with ¹⁴C-labeled carbons at the C-1′, C-2′, C-1, C-2, C-3 or C-4 positions. This is accomplished by utilizing appropriately ¹⁴C-labeled creatine or ethanol starting materials available from American Radiolabeled Chemicals, Inc. of St. Louis, Mo.

Creatine ethyl ester hydrochloride may be produced with ¹³C-labeled carbons at the C-1′, C-2′, C-1, C-2, C-3 or C-4 positions utilizing appropriately ¹³C-labeled creatine or ethanol starting materials as available from Cambridge Isotope Laboratories, Inc. of Andover, Mass.

Creatine ethyl ester hydrochloride may also be labeled by substituting ²H or ³H-labeled hydrogen atoms for hydrogens connected to the C-1′, C-2′, C-C-2 or C-4 positions. Such ²H-labeled creatine or ethanol starting materials are available from Cambridge Isotope Laboratories, Inc. while such ³H-labeled creatine and ethanol starting materials are available from American Radiolabeled Chemicals, Inc.

Finally, creatine ethyl ester hydrochloride may be prepared with nitrogen labeled atoms. More specifically, the N-S, N-6 or N-7 nitrogen atoms may be ¹⁵N-labeled utilizing appropriately labeled creatine starting materials available from Cambridge Isotope Laboratories, Inc.

What is claimed:

1. A compound comprising the chemical formula: 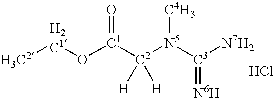
wherein the carbon at any one of the C-1′, C-2′, C-1, C-2, C-3 or C-4 positions is a ¹⁴C-labeled carbon.

2. A compound comprising the chemical formula: 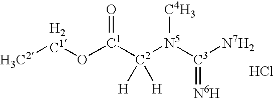
wherein the carbon at any one of the C-1′, C-2′, C-1, C-2, C-3 or C-4 positions is a ¹³C-labeled carbon.

3. A compound comprising the chemical formula: 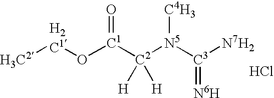
wherein a hydrogen atom connected to any one of the C-1′, C-2′, C-2 or C-4 carbons is an ²H-labeled hydrogen atom.

4. A compound comprising the chemical formula: 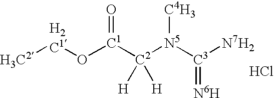
wherein a hydrogen atom connected to any one of the C-1′, C-2′, C-2 or C-4 carbons is an ³H-labeled hydrogen atom.

5. A compound comprising the chemical formula: 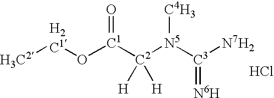
wherein a nitrogen atom at any one of the N-5, N-6 or N-7 positions is a ¹⁵N-labeled nitrogen atom.
